(19) United States
(12) Patent Application Publication
(10) Pub. No.: US 2017/0281965 A1
(43) Pub. Date: Oct. 5, 2017

(54) ASSEMBLY FOR PHOTODYNAMIC THERAPY

(71) Applicant: Tom KERBER, HAMILTON (CA)

(72) Inventor: Tom KERBER, HAMILTON (CA)

(21) Appl. No.: 15/508,119

(22) PCT Filed: Sep. 3, 2015
(86) PCT No.: PCT/IL2015/050889

§ 371 Date: Mar. 2, 2017

Related U.S. Application Data
(60) Provisional application No. 62/046,058, filed on Sep. 4, 2014.

(51) Int. Cl. A61N 5/06

(52) U.S. Cl. CPC A61N 5/062; A61N 5/0616; A61N 2005/005

(57) ABSTRACT

An assembly for photomedicine therapy comprising: (a) at least partially transparent body having an internal cavity; the internal cavity provided with input and output pipes configured for circulating a cooling agent via said internal cavity; and (b) at least one electrically energizable light source disposed within said internal cavity such that radiation from said at least one light source propagates to the ambient surroundings.

### FIELD OF THE INVENTION

The present invention relates to a device for photodynamic therapy, and more particularly to a therapeutic device having decreased output in the far infrared spectrum.

### BACKGROUND OF THE INVENTION

It has been found that in many applications, such as medical healing applications, light can be effective in generally helping the human body to heal itself, and also can be effective in providing energy as a catalyst for various pharmaceutical drugs used in treatment of the human body. Further, it is known that in using light to treat the human body, certain wavelengths of light penetrate readily into to the human body and are therefore available for therapy of tissue in the human body, while other wavelengths of light are quickly and readily absorbed by human skin and the tissue immediately below the skin. More specifically, wavelengths of light below about 1300 nm and above 600 nm tend to pass deeply into the human body, while wavelengths of light above about 1300 nm and below about 600 nm are quickly and readily absorbed by human skin and tissue immediately below the skin, and therefore do not pass deeply into the human body.

It is believed that the attenuation of light energy at wavelengths above about 1300 nm and below about 600 nm is due to the high water content of human tissue. Water typically constitutes about sixty percent of human tissue.

Since light in the range above about 1300 nm and below about 600 nm is very quickly absorbed by human tissue, it inevitably tends to strongly heat and even burn the skin and tissue immediately below the skin, to a point where the heating and burning is unbearable to the person, which is unacceptable in therapy and medical treatment. Further, the fact that a significant number of nerve endings exist in the skin, tends to make this problem even more severe.

Further, this means that the light energy in the range above about 1300 nm and below about 600 nm is not being transmitted deeply into the human body tissue where it is needed. Accordingly, it is necessary to have either less intense light, which is unacceptable since very little light energy would pass deeply into the tissue, or to produce light that has wavelengths only below 1300 nm and above about 600 nm, since light in this range is not attenuated as readily by the human body.

It should be noted that glass typically tends to pass light in the frequency range having wavelengths below 3000 nm Accordingly, a light/lamp typically has a light output below about 3000 nm.

It should also be noted that halogen lights produce light from about 400 nm all the way to perhaps 3000 nm, and have a peak output slightly below 1000 nm Accordingly, more than half of the light energy of a halogen light is in the frequency range that is readily absorbed by the skin and tissue of the human body. Therefore, use of an unfiltered halogen light to treat the human body would produce a very significant amount of heat and would quickly heat the skin to unacceptably high levels, thus causing pain and even “burning” of the skin.

It is also well-known that in many applications where intense light is used, such as medical operating rooms, theatre lighting, and so on, it would be beneficial to reduce the amount of heat generated from such lights. This heat is generated substantially from the wavelengths of light in the frequency range above 1000 nm and below about 400 nm, since heat is perceived by a person and is due to the absorption of light energy by the skin.

One such prior art assembly for photodynamic therapy, developed by the present inventor, comprises a light assembly having reduced heat output. The light assembly comprises a light emitting main body having a substantially hollow interior. An aqueous solution is disposed within the substantially hollow interior of the light emitting main body. An ingress in the light emitting main body is in fluid communication with the substantially hollow interior, to permit the ingress of the aqueous solution into the substantially hollow interior of the light emitting main body. An egress in the light emitting main body is in fluid communication with the substantially hollow interior, to permit the egress of the aqueous solution from the substantially hollow interior of the light emitting main body. The light emitting main body is otherwise sealed to preclude the escape of the aqueous solution therefrom. The light emitting main body includes a first substantially transparent window and a second substantially transparent window. The first substantially transparent window and the second substantially transparent window are aligned such that at least some of the light that enters the substantially hollow interior of the light emitting main body through the first substantially transparent window, passes through the aqueous solution and also exits the substantially hollow interior of the light emitting main body through the second substantially transparent window. A source of light is operatively mounted on the light emitting main body so as to transmit light to the first substantially transparent window, such that the transmitted light generally passes through the first substantially transparent window, the aqueous solution, and the second substantially transparent window. The above discussed assembly for photodynamic therapy and the technology related to it are disclosed at http://www.genesishealthlight.com/science/wavelength-output-power. Although the above discussed therapy light works well, it has a maximum usable output of about fifteen (15) therapeutic watts. Above this level of intensity, the unit is not capable of discharging the excess heat and will shut down due to thermal overload. Potentially, the plastic body of the unit may also melt. Accordingly, the above discussed assembly for photodynamic therapy is not as effective as is needed for treating deep tissue injuries.

A prior art light that deals with the filtration of light in the food industry, is disclosed in U.S. Pat. No. 5,832,441, issued Jan. 17, 1995 to Lentz et al., and entitled Method Of Processing Food Utilizing Infrared Radiation. The disclosed method includes a method of selectively heating, comprising the step of providing a source of radiation capable of delivering at least 60% of its power in a selected wavelength band, and exposing the food to the radiation for an amount of time sufficient to heat beneath a surface. The Lentz et al. patent teaches the use of a filtered light for the purpose of use with food only. It also teaches that the maximum penetration occurs at about 1,000 nanometers, and the depth of penetration corresponding to the maximum is about 0.47 centimeters, which is not a significant depth in terms of treating human tissue.

It is an object of the present invention to provide an assembly for photomedicine therapy having reduced heat output.

It is an object of the present invention to provide a very quiet assembly for photomedicine therapy having reduced heat output.

It is an object of the present invention to provide a very quiet assembly for photomedicine therapy having reduced heat output that has no fan in the light emitting main body.

It is another object of the present invention to provide an assembly for photomedicine therapy having reduced heat output, wherein the light is usable as a medical assembly for photomedicine therapy.

It is another object of the present invention to provide an assembly for photomedicine therapy having reduced heat output, wherein the light is usable as a medical treatment light.

It is an object of the present invention to provide an assembly for photomedicine therapy having reduced heat output, and having significantly greater therapeutic light intensity than prior art assembly for photomedicine therapy.

It is a further object of the present invention to provide an assembly for photomedicine therapy having reduced heat output, wherein the assembly for photomedicine therapy is small and light-weight.

### SUMMARY OF THE INVENTION

It is hence one object of the invention to disclose an assembly for photomedicine therapy comprising: (a) a body having an internal cavity; said internal cavity is provided with input and output pipes configured for circulating a cooling agent via said internal cavity; (b) at least one electrically energizable light source disposed within said internal cavity such that radiation from said at least one light source propagates to the ambient surroundings.

Another object of the invention is to disclose the cooling agent which is an aqueous solution or a non-aqueous solution.

A further object of the invention is to disclose the solution further comprises a colouring matter.

A further object of the invention is to disclose a system for photomedicine therapy comprising: (a) at least partially transparent body having an internal cavity; said internal cavity is provided with input and output pipes configured for circulating a cooling agent via said internal cavity; (b) at least one electrically energizable light source disposed within said internal cavity of said body such that radiation from said at least one electrically energizable light source of light propagates to the ambient surroundings;(c) a cooling circuit further comprising a pump and heat radiator fluidly connected with said internal cavity in series.

A further object of the invention is to disclose the cooling agent which is an aqueous solution or a non-aqueous solution.

A further object of the invention is to disclose the solution further comprises a colouring matter.

A further object of the invention is to disclose a filtering arrangement for a photomedicine therapy having comprising at least partially transparent body having an internal cavity. The aforesaid cavity is configured for mounting a light source. The internal cavity is provided with input and output pipes configured for circulating a cooling agent via said internal cavity,

It is a core purpose of the invention to provide the cooling agent comprising a colouring matter absorbing visible light (400-600 nm) and aqueous solution absorbing infrared radiation (1300-5000) nm.

A further object of the invention is to disclose a method of providing photomedicine therapy to a patient, said method comprising the steps of: (a) providing an assembly for photomedicine therapy comprising: (i) at least partially transparent body having an internal cavity; said internal cavity is provided with input and output pipes configured for circulating a cooling agent via said internal cavity; (ii) at least one electrically energizable source of light disposed within said cavity of said body such that light from said at least one electrically powerable source of light passes to the ambient surroundings; (b) positioning said assembly adjacent a patient; (c) illuminating said assembly for a period of time; and, (d) circulating a coloured cooling agent within said body. The power density provided by the light source in a treatment plane is from 0.1 watts/cm² to 2 watts/cm², over an area of 60 cm²

Other advantages, features and characteristics of the present invention, as well as methods of operation and functions of the related elements of the structure, and the combination of parts and economies of manufacture, will become more apparent upon consideration of the following detailed description and the appended claims with reference to the accompanying drawings, the latter of which is briefly described herein below.

### BRIEF DESCRIPTION OF THE DRAWINGS

The novel features which are believed to be characteristic of the assembly for photomedicine therapy according to the present invention, as to its structure, organization, use and method of operation, together with further objectives and advantages thereof, will be better understood from the following drawings in which a presently preferred embodiment of the invention will now be illustrated by way of example. It is expressly understood, however, that the drawings are for the purpose of illustration and description only, and are not intended as a definition of the limits of the invention. In the accompanying drawings:

FIGS. 1 and 2 are side and front schematic views of a first embodiment of the assembly for photomedicine therapy;

FIG. 3 is a sectional schematic view of a second illustrated embodiment of the assembly for photomedicine therapy; and

FIG. 4 is a schematic view an external cooling system.

### DETAILED DESCRIPTION OF THE ILLUSTRATED EMBODIMENTS

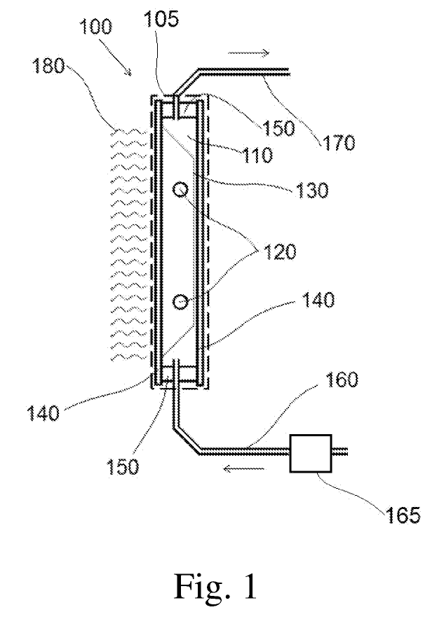

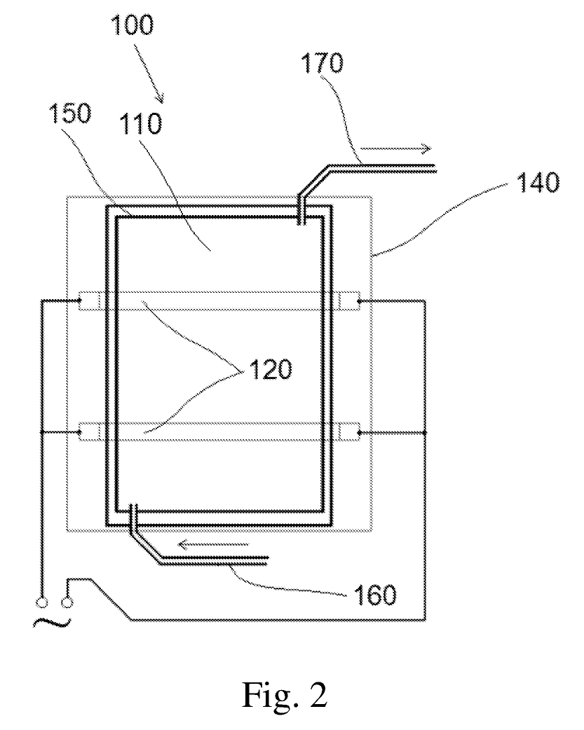

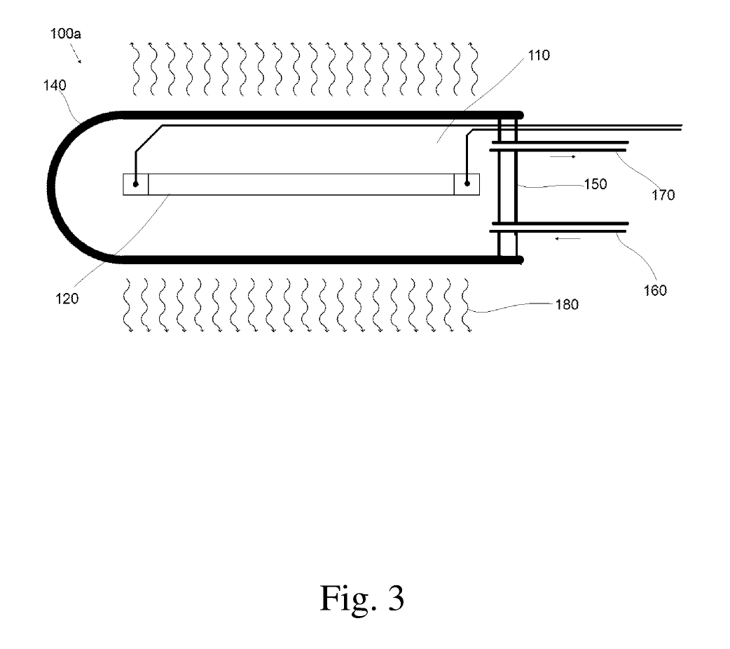

Referring to FIGS. 1 through 3 of the drawings, it will be noted that FIGS. 1 and 2 illustrate a first illustrated embodiment of the assembly for photomedicine therapy according to a first embodiment of the present invention, and FIG. 3 illustrates a second embodiment of the assembly for photomedicine therapy according to the present invention. The assembly is equally applicable to humans and animals.

Reference is now made to FIGS. 1 and 2, showing a first embodiment of assembly for photomedicine therapy 100 which comprises a body 105 having an internal cavity 110. The aforesaid cavity 110 is defined glass enclosure 140 where at least one halogen lamp 120 is mounted. Directing treating radiation 180 is assisted by reflector 130. Internal cavity 110 is sealed by silicon members 150. The aforesaid internal cavity is cooled by a cooling agent (not shown) circulating through cavity 110. The cooling system belonging to assembly 100 for photomedicine therapy is not shown in FIGS. 1 to 3 and will be described in detail below. The cooling agent is fed through inlet pipe 160 and exhausted through outlet pipe 170. A specific shape of the body 105 is dictated by a specific use and can be conformable to a configuration of an organ or a body part to be treated. Halogen lamp 120 is mounted within body 105 by means of standard bolting or other fastening means and energized by a source of electricity.

According to one embodiment of the present invention, assembly 100 for photomedicine therapy comprises valve 165 which is manually operable and used for controlling the flow of the cooling agent through cavity 110 of body 105 and close loop cooling system connected thereto (not shown).

Reference is now made to FIG. 3 presenting the second embodiment of the present invention constituting probe 100a where the glass enclosure 140 is configured as an elongate glass cover. Assembly 100a in the form of a probe can be used either externally adjacent human skin, or internally to exemplarily treat gum disease, an enlarged prostate, or uterine disorders.

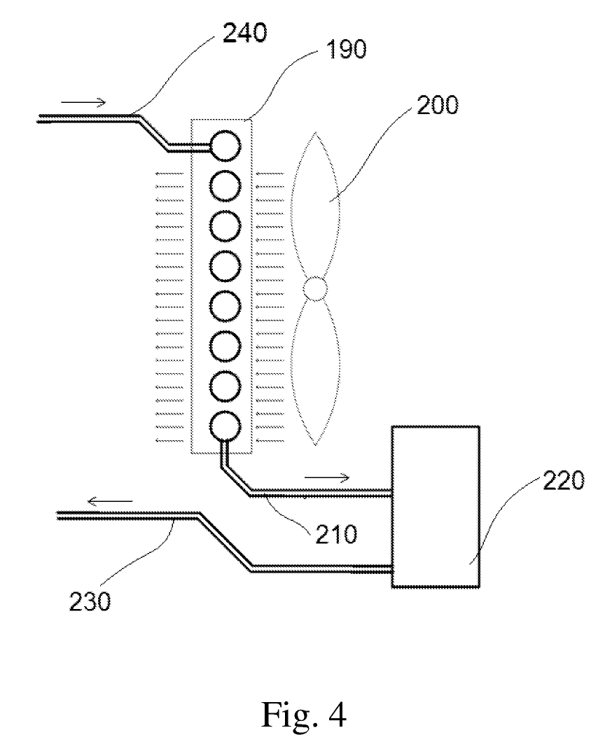

Reference is now made to FIG. 4 showing a cooling system which is a portion of described above assembly 100 for photomedicine therapy. A cooling agent circulates within a cooling circuit including body 105, radiator 190, pump 220 which are connected to each other in series by means of pipes 240, 210 and 230. Radiator 190 is force-cooled by fan 200. Preferably, the cooling agent is an aqueous solution of colouring matter absorbing the lamp radiation at undesirable spectral bands (for example, at blue, green and yellow wavelengths). This combination has been found to work well, and is also inexpensive and readily available. The cooling agent is in heat conductive relation with the at least one electrically energized light source 120, for example, the halogen lamp 120, so as to cool the halogen lamp 120 during normal operation.

In use, assembly 100 preferably provided with halogen lamp 120, is energized by closing an electrical switch and or intensity control (raising or lowering voltage) (not specifically shown). Assembly 100 has an emission spectrum of between about 400 nm and about (or beyond) 3000 nm The light from the halogen lamp 120 is filtered by the cooling agent such that light having wavelengths in the range of about 600 to about 1300 nm passes through cavity 110 filled with the cooling agent and then passes through the elongate glass cover 140 to reach a patient for treatment. It has been found that with assembly 100 according to the present invention, the filtered light can penetrate approximately 6-8 centimetres into human tissue. Further, assembly 100a does not cause any burning of the skin of a patient, even with the use of a three hundred (300) watt halogen lamp 120 when assembly for photomedicine therapy 100 is adjacent the patient's skin.

It should also be noted that assembly 100 for photomedicine therapy according to the present invention is extremely quiet during use as there is no fan used in body 105.

As can be understood from the above description and from the accompanying drawings, the present invention provides an assembly for photomedicine therapy having reduced heat output, a very quiet assembly for photomedicine therapy having reduced heat output, a very quiet assembly for photomedicine therapy having reduced heat output that has no fan in the light emitting main body, wherein the assembly for photomedicine therapy is usable as a medical assembly for photomedicine therapy, wherein the assembly for photomedicine therapy is usable as a medical treatment light, that has significantly greater therapeutic light intensity than prior art assembly for photomedicine therapy, and wherein the assembly for photomedicine therapy is small and light-weight, all of which features are unknown in the prior art.

Other variations of the above principles will be apparent to those who are knowledgeable in the field of the invention, and such variations are considered to be within the scope of the present invention. For instance, the light assembly could comprise something more similar to a light bulb, or alternatively could be made from flexible plastic to enable it to be placed on the arm, leg or torso of a person, for instance. Further, other modifications and alterations may be used in the design and manufacture of the light assembly of the present invention without departing from the spirit and scope of the accompanying claims.

1.-8. (canceled)

9. An assembly for photomedicine therapy comprising:
a. at least partially transparent body having an internal cavity; said internal cavity is provided with input and output pipes configured for circulating a cooling agent through a pump and a heat radiator via said internal cavity in a close-loop manner;
b. at least one electrically energizable halogen lamp disposed within said internal cavity containing said cooling agent such that radiation from said at least one halogen lamp propagates to the ambient surroundings through said cooling agent while cooling said halogen lamp.

10. The assembly according to claim 9, wherein said cooling agent is an aqueous solution or a non-aqueous solution.

11. The assembly according to claim 10, wherein said solution further comprises a colouring matter.

12. A system for photomedicine therapy comprising:
a. at least partially transparent body having an internal cavity; said internal cavity is provided with input and output pipes configured for circulating a cooling agent via said internal cavity;
b. at least one electrically energizable halogen lamp disposed within said internal cavity of said body containing said cooling agent such that radiation from said at least one electrically energizable—halogen lamp propagates to the ambient surroundings through said cooling agent while cooling said halogen lamp;
c. a cooling circuit further comprising a pump and heat radiator fluidly connected with said internal cavity in a close-loop manner.

13. The assembly according to claim 12, wherein said cooling agent is an aqueous solution or a non-aqueous solution.

14. The assembly according to claim 12, wherein said solution further comprises a colouring matter.

15. A method of providing photomedicine therapy to a patient, said method comprising the steps of:
a. providing an assembly for photomedicine therapy comprising:
i. at least partially transparent body having an internal cavity; said cavity configured for mounting a halogen lamp; said internal cavity is provided with input and output pipes configured for circulating a cooling agent through a pump and a heat radiator via said internal cavity in a close-loop manner;
ii. at least one electrically energizable halogen lamp disposed within said cavity such that light from said at least one electrically powerable halogen lamp passes to the ambient surroundings through said cooling agent while cooling said halogen lamp;
b. positioning said assembly adjacent a patient;
c. illuminating said assembly for a period of time; and,
d. circulating a coloured cooling agent within said body;
wherein power density provided by the light source in a treatment plane is from 0.1 watts/cm² to 2.0 watts/cm² over an area of 60 cm².

16. The assembly according to claim 9, wherein said cooling agent is configured to filter wavelengths below 600 nm and above 1300 nm.

17. The assembly according to claim 9, wherein the cooling agent comprises a colouring matter configured to filter undesirable spectral bands at blue, green and yellow wavelengths.
